(19) United States
(12) Patent Application Publication
(10) Pub. No.: US 2026/0185126 A1
(43) Pub. Date: Jul. 2, 2026

(54) Pharmaceutical compositions/medications made from White Tumeric (Curcuma Aromatica Salisb.) compounds having enhanced inhibitory urease enzyme activities and methods of manufacturing the same

(71) Applicants: THANH MAI THI NGUYEN, HO CHI MINH (VN); NHAN TRUNG NGUYEN, HO CHI MINH (VN); THO HUU LE, HO CHI MINH (VN); NHAT TRUONG VAN DO, HO CHI MINH (VN); HAI XUAN NGUYEN, HO CHI MINH (VN)

(72) Inventors: THANH MAI THI NGUYEN, HO CHI MINH (VN); NHAN TRUNG NGUYEN, HO CHI MINH (VN); THO HUU LE, HO CHI MINH (VN); NHAT TRUONG VAN DO, HO CHI MINH (VN); HAI XUAN NGUYEN, HO CHI MINH (VN)

(73) Assignees: VietNam National University Ho Chi Minh City, HO CHI MINH (VN); VNUHCM-University of Science, HO CHI MINH (VN)

(21) Appl. No.: 19/051,237

(22) Filed: Feb. 12, 2025

(30) Foreign Application Priority Data
Dec. 27, 2024 (VN) 1-2024-09776
Dec. 27, 2024 (VN) 1-2024-09777
Dec. 27, 2024 (VN) 1-2024-09778
Dec. 27, 2024 (VN) 1-2024-09779

(51) Int. Cl. C12P 7/18; C07C 39/12; C07C 69/21

(52) U.S. Cl. CPC C12P 7/18; C07C 39/12; C07C 69/21

(57) ABSTRACT

A method and a composition, characterized as having enhanced inhibitory urease activities, having a diarylheptanoid compound having a first aryl moiety group coherently bonded to a linear heptane moiety and a second aryl moiety group described by the following formula:

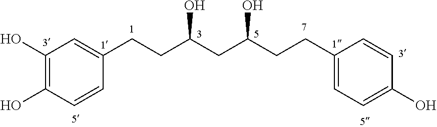 [SMILES: Oc1ccc(CC[C@H](O)C[C@H](O)CCc2ccc(O)c(O)c2)cc1]

### CROSS-REFERENCE TO RELATED APPLICATIONS

This application claims priority under 35 U.S.C. § 119(a) of the patent application No. 1-2022-07433, entitled “ [symbol] Pháp, [symbol] Th\hat{ó}ng [symbol] Lý [symbol] [symbol] S\hat{ó} [symbol] [symbol] [symbol] [symbol] [symbol] Trên N\hat{è}n Chu\hat{õ}i Kh\hat{ó}i”, by the same inventor Tung Thanh Tran, filed on Nov. 14, 2022 in the Republic Socialist of Vietnam. The patent application identified above is incorporated in its entirety herein to provide continuity of disclosure.

### FIELD OF THE INVENTION

The present invention relates generally to pharmaceutical compositions/medications for the treatment of gastric ulcers. More specifically, the present invention relates to methods and pharmaceutical compositions derived from white turmeric rhizomes (Curcuma Aromatica Salisb.).

### BACKGROUND ART

Gastrointestinal illnesses including peptic ulcer is a condition in which open sores develop in the stomach lining (gastric ulcers) or the small first part of the intestine (duodenal ulcers). The pathophysiology of gastrointestinal illnesses has been proven to be associated with the infection of Helicobacter pylori (H. pylori) in many studies.¹ It has been shown that H. pylori secrete urease enzyme that plays a crucial role in colonizing and surviving the bacteria in the stomach.² The secretion of urease reduces the mucosal defense mechanism of the stomach, decreasing the ability of the stomach to make acid (achlohydia). Urease is a nickel-dependent enzyme, produced by plants, fungi, and bacteria but not animals. It hydrolyzes urea into ammonia (NH₃) and carbon dioxide (CO₂), neutralizing the acidic environment of the stomach and promoting bacterial colonization.³ Because of its vital role in gastrointestinal illnesses, urease is an attractive target for developing drugs against gastrointestinal illnesses induced by H. pylori.⁴

The structure of this enzyme was first revealed through the crystallized structure of jack-bean urease (JBU), showing that the JBU structure adopts a special T-shape of a hammer consisting of four domains with the active site occupied by two nickel atoms. The urease structures known up to date are found to share significant homology, resulting in the similar architecture of the active site and similar mechanism of catalysis.^{7, 8} The active site of urease is characterized as a pocket covered by a 30-residue helix-turn-helix motif, also called a mobile flap, which works as a gate for the substrate influx and product efflux when it opens.^{9, 10} To interfere with the urea hydrolysis reaction catalyzed by urease, a variety of both competitive and non-competitive urease inhibitors have been developed, including hydroxyurea, thiourea, acetohydroxamic acid (AHA), phosphoramidites, quinones, and Au(III) compound, and so on.¹¹⁻²⁶ Among the known urease inhibitors, AHA and hydroxyurea are the substrate analog inhibitors. The common characteristics of their binding mechanism are that each of their carbonyl (C═O) oxygen atoms is primarily coordinated with the Ni¹ ion, and the Ni¹-bound oxygen atom forms a hydrogen bond with a histidine residue nearby in the active site of the enzyme.²⁷ Since most of these currently used inhibitors are either too toxic or unstable,²² the design of novel urease inhibitors with enhanced strength is currently in demand.

In one attempt, urease inhibitor compounds were extracted from plants are used to reduce urease activities. A. Wajid Khan and colleagues reported that the compound (E)-N-(4-hydroxy-3-methoxyphenethyl)-3-(4-hydroxy-3-ethoxyphenyl) acryl amide has anti-gastric ulcer activity with % inhibition value of 64.6±4.2 at a concentration of 0.2 mg/mL. In another attempt, Thiourea was used as a positive control with % inhibition value of 98.2±4.3 at a concentration of 0.2 mg/mL. (Khan A. W., Jan S., Parveen S., Khan R. A., Saeed A., Tanveer A. J., Shad A. A., Phytochemical analysis and enzyme inhibition Assay of Aerva javanica for Ulcer. (Chemistry Central Journal, 6(1), 1-6, 2012). Woo-Yong Jeon and his colleagues used white turmeric to treat gastritis on mice. However, this method only yielded limited urease inhibitory activities and only in the laboratory scale only. The primary cause of the treatment failure against H. pylori infection is the antibiotic resistance of the bacteria. The antibiotic resistance renders the antibiotic treatments ineffective and bioavailability insufficient.

Therefore, there is a need for compounds with enhanced urease inhibitory activities and sufficient bioavailability.

There is a need for a method of synthesizing compounds with enhanced urease inhibitory activities and sufficient bioavailability.

There is a need for pharmaceutical compositions or medications having enhanced urease inhibitory activities that effectively treat gastrointestinal illnesses.

There is a need for methods for manufacturing pharmaceutical compositions or medications with enhanced urease inhibitory activities that have harmless to human and sufficient bioavailability.

The methods and pharmaceutical compositions/medications of the present invention meets the above needs and solve the above-described requirements.

### SUMMARY OF THE INVENTION

Accordingly, an object of the present invention is to provide compounds extracted from plants or natural resources that have enhanced inhibitory urease activities that effectively treat gastrointestinal illnesses.

Another object of the present invention is to provide a method for treating gastrointestinal illnesses including gastritis, peptic ulcers, and gastric cancers using a compound with enhanced inhibitory urease activities and bioavailability.

Another object of the present invention is to provide pharmaceutical compositions or medications for treating gastrointestinal illnesses with effectiveness, non-toxicity, and sufficient bioavailability.

Another object of the present invention is to provide methods for manufacturing pharmaceutical compositions or medications for effectively treating gastrointestinal illnesses.

Another object of the present invention is to provide a method and a composition, extracted from white turmeric (Turmeric Aromatica Salisb.) characterized as having enhanced inhibitory urease activities, having a diarylheptanoid compound described by the following formula:

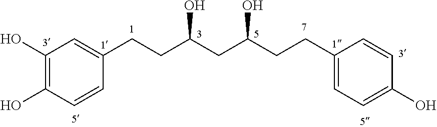 [SMILES: Oc1ccc(CC[C@H](O)C[C@H](O)CCc2ccc(O)c(O)c2)cc1]

These and other advantages of the present invention will no doubt become obvious to those of ordinary skill in the art after having read the following detailed description of the preferred embodiments, which are illustrated in the various drawing and figures.

### BRIEF DESCRIPTION OF THE DRAWINGS

The accompanying drawings, which are incorporated in and form a part of this specification, illustrate embodiments of the invention and, together with the description, explain the principles of the invention.

FIG. 1 is a flowchart of a method of using pharmaceutical compositions/medications using enhanced inhibitory urease activities of compounds extracted from white turmeric in accordance with an exemplary aspect of the present invention;

FIG. 2 is a flowchart of a method for manufacturing white turmeric based compounds with enhanced inhibitory urease activities in accordance with an exemplary embodiment of the present invention;

FIG. 3 illustrates chemical formulas of new sub-fractions from white turmeric extraction in accordance with an exemplary aspect of the present invention;

FIG. 4 presents chemical formulas of three new sub-fractions extracted from white turmeric rhizomes in accordance with an exemplary aspect of the present invention;

FIG. 5 illustrates binding poses and interactions of sub-fraction 4(R)—(S) in the active sites of urease in accordance with an exemplary aspect of the present invention;

FIG. 6 illustrates binding poses and interactions of sub-fraction 4(S)—(R) in the active sites of urease in accordance with an exemplary aspect of the present invention;

FIG. 7 illustrates binding poses and interactions of sub-fraction 9(R)—(S) in the active sites of urease in accordance with an exemplary aspect of the present invention;

FIG. 8 illustrates binding poses and interactions of sub-fraction 9(S)—(R) in the active sites of urease in accordance with an exemplary aspect of the present invention; and

FIG. 9 illustrates chemical formula for sub-fraction No. 4 that are used in the treatment of gastrointestinal illnesses in accordance with an aspect of the present invention.

The above figures are for the purposes of illustration only. A person of ordinary skill in the art will readily recognize from the following discussion that alternative embodiments of the structures and methods illustrated herein may be employed without departing from the principles of the technology described herein.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

Within the scope of the present description, the term, “a compound with enhanced urease inhibitory activities” means the compound having potency against the Helicobacter pylori (H. pylori) bacteria. In other words, the compound with enhanced urease inhibitory activities has the highest IC₅₀. IC₅₀ is defined as half-maximal inhibitory concentration. The compound with enhanced urease inhibitory activities is also defined by the inhibitory percentage (I % of the samples was calculated using the following equation:

$$I \% = \frac{{A}_{0} - {A}_{1}}{{A}_{0}} \times 100 \% ,$$

where I is inhibition, A₀ and A₁ represent the activities of the enzyme without and with the test sample, respectively. The enzyme inhibitory percentage is used to determine the activity of the samples in the above assay. IC₅₀ values are determined from the mean data obtained from these experiments for the tested activities.

Within the scope of the present description, the words “pharmaceutical compositions”, “medications”, “pills”, “medicines”, “medicaments”, “compounds”, “solutions”, “formulations”, or “drugs” all mean medicinal substance made to cure gastrointestinal illnesses caused by the Helicobacter pylori (H. pylori) bacteria.

Within the scope of the present description, the words drug administration means all methods used to inject the pharmaceutical compositions of the present invention to the patients who suffer from gastrointestinal illnesses. The drug administration includes, but not limited to, oral, intravenous, rectal routes, injection, enteral, etc. that are most effective to treat the gastrointestinal illnesses.

Within the scope of the present invention, reference to “an embodiment” or “the embodiment” or “some embodiments” means that a particular feature, structure, or element described with reference to an embodiment is comprised in at least one embodiment of the described object. The sentences “in an embodiment,” “in the embodiment,” or “in some embodiments” in the description do not, therefore, necessarily refer to the same embodiment or embodiments. The features, structures, or elements can be furthermore combined in any adequate way in one or more embodiments.

The present invention provides a method for treating gastric ulcers or other gastrointestinal illnesses using plant-based diarylheptanoid substances characterized in having maximal inhibitory urease activities.

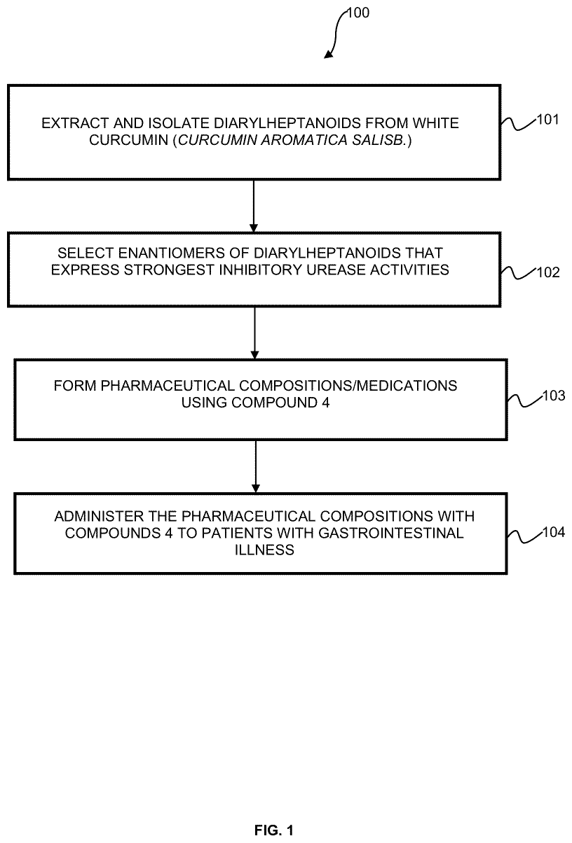

Now referring to FIG. 1, a flowchart of a process 100 for synthesizing pharmaceutical compositions/medications designed to treat gastrointestinal illnesses caused by p. Hilerba (Helicobacter pylori (H. pylori) in accordance with an exemplary embodiment of the present invention is illustrated. Process 100 involves designing the enhanced inhibitory urease activities from plant-based compounds. In addition, method 100 uses only chemical compounds extracted from plants and vegetation in such a novel manner that it can be used in industrial scale.

At step 101, diarylheptanoid compounds are isolated and extracted from white turmeric (Turmeric aromatic Salibs.) rhizomes. Step 101 is realized by Soxhlet extraction method. The Soxhlet extraction method uses the Soxhlet extractor which uses the siphon principle and solvent reflux to extract the solid white turmeric powder with pure solvent. The white turmeric powder is placed in a holder called “thimble” inside the main chamber of the Soxhlet extractor. By refluxing the Methanol (MeOH) solvent through the thimble using a condenser and a siphon side arm, the extraction cycle is repeated many times. Organic solvent such as methanol (MeOH or CH₃OH) is generally selected along with acidic or basic residues. The amount of solvent keeps rising till the overflow level beyond which it is siphoned to the distillation flask. This introduces the extracted analyses to the bulk solution from which the solvent vapors emanate due to elevated temperatures. It is noted that other extraction methods such as reflux extraction, steam distillation, ultrasound extraction, microwave-assisted extraction, supercritical fluid extraction, pressurized liquid extraction, enzyme-assisted, ionic liquid extraction, etc. can be used to realize step 101. All of these listed methods are within the scope of the present invention and available in the market that they need not to be described in details herewith.

Step 101 further includes separation and identification to obtain and identify sub-fractions contained in the harvested white turmeric rhizomes. In many aspects of the present invention, column chromatography is used for separation step. Identification step uses spectroscopy methods to identify the chemical compounds of the white turmeric. The chemical compounds of white turmeric includes alkaloids, terpenoids, flavonoids, steroids, saponins, tannins, phenols, phytosterols, glycosides, protein amino acids, and volatile essential oils. Some diarylheptanoid compounds in turmeric include turmeric; demethoxyturmeric; aromaticanoid A; aromaticanoid B; aromaticanoid C and aromaticanoid D; curcumaromin A; curcumaromin B; curcumaromin C; (3S,5S)-dihydroxy-1,7-bis(3,4-dihydroxyphenyl)heptane; (3S,5S)-dihydroxy-1-(3-hydroxy-4-methoxyphenyl)-7-(4-methoxyphenyl)heptane; (3S,5S)-dihydroxy-1-(4-methoxyphenyl)-7-phenylheptane; (3R,5R)-dihydroxy-1-(3,4-dihydroxyphenyl)-7-(4-hydroxyphenyl)heptane and 2,3,5-trihydroxy-1,7-bis(4-hydroxy-3-methoxyphenyl)heptane.

Next at step 102, sub-fractions having enhanced inhibitory urease activities are derived the sub-fractions obtained from step 101. In many aspects of the present invention, step 102 is realized by structure activity relationship (SAR), molecular docking, and electronic structure computations. SAR model provides relation between the chemical structure of the molecule and therapeutic efficacy. In other words, SAR analysis provides justification about the most effective chemical (functional) group which triggers the therapeutic to the next level. Three dimensional (3D) SAR model capable of investigating stereochemistry and compounds that depend on chirality. A software program from StarDrop's Glowing Molecule™ is used to build 3D SAR models. Molecular docking is a computational technique that predicts the binding affinity of ligands to receptor proteins. Software programs such as DOCK 3.5×, Autodock Vinaptimal, Argus lab 4.0.1, Genetic Optimization for Ligand Docking (GOLD™), etc. can be used to realize step 102. Electronic structure computations enable advanced computations spanning from high-level electronic structure calculations over molecular simulations to machine learning. These software are open-source provided by Computational Chemical Sciences (CCS) which include AutoTST, Chemical VAE, CP2K, CSPib, DeePMD-kit, EMSL Arrows, FLOSIC18, GAMESS, etc.

Next, at step 103, pharmaceutical compositions/medications against gastrointestinal illnesses caused by Helicobacter pylori (H. pylori) are manufactured using the sub-fraction having enhanced inhibitory urease activities obtained from step 102. In many aspects of the present invention, step 103 is realized by tablets and/or liquid medications pharmaceutical processes. Pharmaceutical medication using the compound with the enhanced inhibitory enzyme activities obtained from step 102 as active pharmaceutical ingredient (API). The API is defined by the following chemical formula. The derivation of the present chemical formula is explained later.

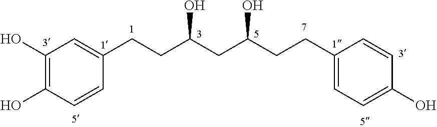 [SMILES: Oc1ccc(CC[C@H](O)C[C@H](O)CCc2ccc(O)c(O)c2)cc1]

Step 103 includes blending, granulation, drying, compression, coating, and polishing. In the blending step, active pharmaceutical ingredient (API) is defined by the chemical formula above is mixed with an excipient. Excipient is the inactive ingredient which serves as a medium for conveying the active ingredient. Within the scope of the present invention, some excipients with a safety impact are identified such as sucrose, saccharin, aspartame, sorbitol, mannitol, lactose, ethanol, propylene glycol, parabens, menthol and silica. In other aspects of the present invention, nano excipients such as metal organic frameworks (MOF) are also used.

Finally, at step 104, the pharmaceutical compositions or medications are administered to patients suffered from gastrointestinal illnesses. Step 104 is realized by injection, oral, intravenous, rectal routes, enteral, sublingual, etc.

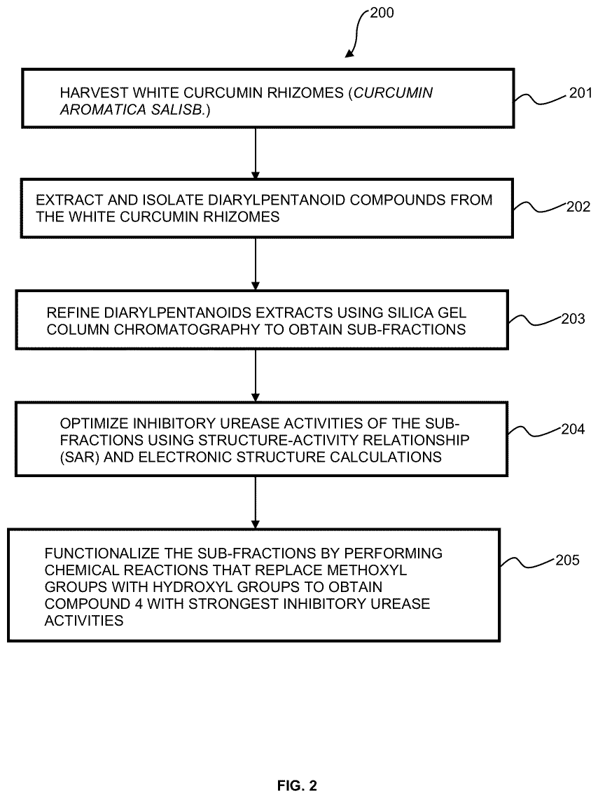

Next, referring to FIG. 2, a method 200 for synthesizing the sub-fraction having enhanced inhibitory urease activities in accordance with an exemplary aspect of the present invention is illustrated.

At step 201, white turmeric rhizomes are harvested. Step 201 is realized by harvesting, drying, and grinding white turmeric rhizomes. White turmeric plants are grown throughout Vietnam.

At step 202, diarylheptanoid compounds are isolated and extracted. The desiccated rhizomes of C. aromatica from step 201 are subjected to extraction using a Soxhlet extractor with methanol (MeOH, CH₃OH, or methyl alcohol) as the solvent. The resulting extracts is then suspended in water and successfully partitioned with n-hexane to eliminate fatty compounds, followed by chloroform to obtain the CHCl₃ extracts. The CHCl₃-soluble extracts undergone further refinement through silica gel column chromatography, employing acetone-CHCl₃ gradient mixtures as eluents, isolating seventeen fractions.

At step 203, sub-fractions of diarylheptanoid compounds are extracted using column chromatography. As described above, column chromatography consists of a stationary solid phase that adsorbs and separates the white turmeric compounds passing through it with the help of a liquid mobile phase. On the basis of the chemical nature of white turmeric, sub-fractions get adsorbed and elution is based on different adsorption characteristics of a substance by the adsorbent.

At step 204, inhibitory urease activities of the sub-fractions are modeled and optimized using structure activity relationship (SAR), molecular docking, and electronic structure. In many aspects of the present invention, step 204 is realized by structure activity relationship (SAR), molecular docking, and electronic structure computations. SAR model provides relation between the chemical structure of the molecule and therapeutic efficacy. In other words, SAR analysis provides justification about the most effective chemical (functional) group which triggers the therapeutic to the next level. Three dimensional (3D) SAR model capable of investigating stereochemistry and compounds that depend on chirality. A software program from StarDrop's Glowing Molecule™ can be used to build 3D SAR models. Molecular docking is a computational technique that predicts the binding affinity of ligands to receptor proteins. Software programs such as DOCK 3.5×, Autodock, Argus lab 4.0.1, Genetic Optimization for Ligand Docking (GOLD™), etc. can be used to realize step 102. Electronic structure computations enable advanced computations spanning from high-level electronic structure calculations over molecular simulations to machine learning. These software are open-source provided by Computational Chemical Sciences (CCS) which include AutoTST, Chemical VAE, CP2K, CSPib, DeePMD-kit, EMSL Arrows, FLOSIC18, GAMESS, etc.

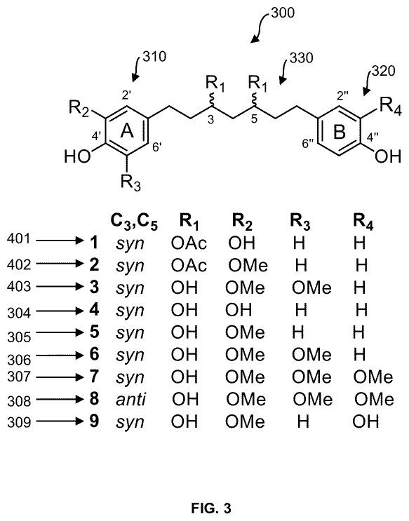

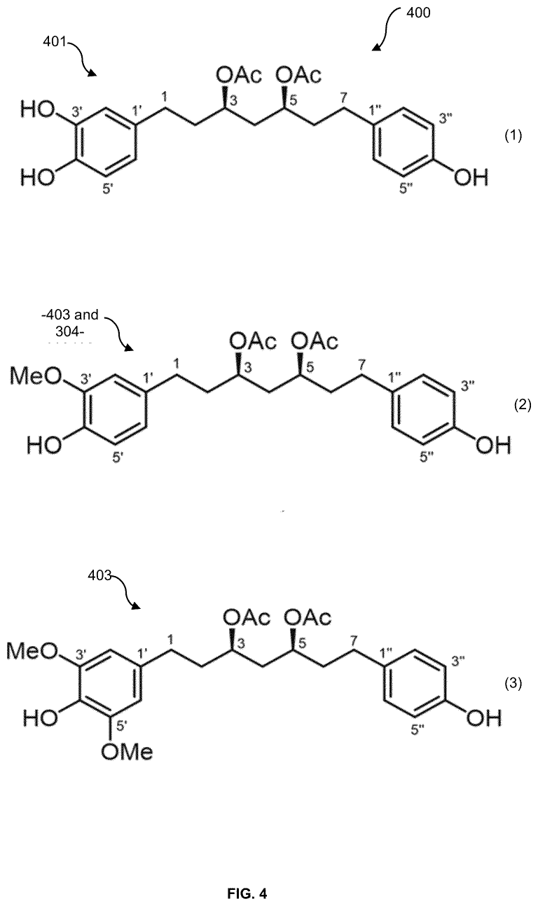

Based on the result of step 204, at step 205, chemical reactions between sub-fractions from step 203 and other reactants are designed and performed to obtain the sub-fraction having the strongest inhibitory urease activities. In many aspects of the present invention, the chemical reactions are designed so that the hydroxyl functional groups (OH) replace and bonds with the linear heptane moiety at positions 3,5. The chemical reactions are performed as follows: After the compounds of the white turmeric are extracted and isolated as described in step 202, three new compounds as described in FIG. 3 and FIG. 4 are obtained. Please refer to FIG. 3 and FIG. 4 for the description of a compound 401. A compound 401 is selected for the following reasons: (1) The amount of compound 401 extracted from methanol (MeOH) of the white turmeric is 1.83%, (2) White turmeric and compound 401 are abundant and can be used in the industrial scale. Hydrochloride acid (HCl) of 36.5% strength is reacted with methanol (MeOH). The weight or volume ratio (% w/w or % v/v) between MeOH and HCl is 30:1. The weight or volume (% w/w or % v/v) between compound 401 and methanol is 10:3. A deacetylation reaction is performed by stirring the mixture at 200 rounds per minute at room temperature and for 24 hours. After this reaction time, compound 401 is completely deacetylized. The deacetylation reaction for compound 401 is by the following reactions:

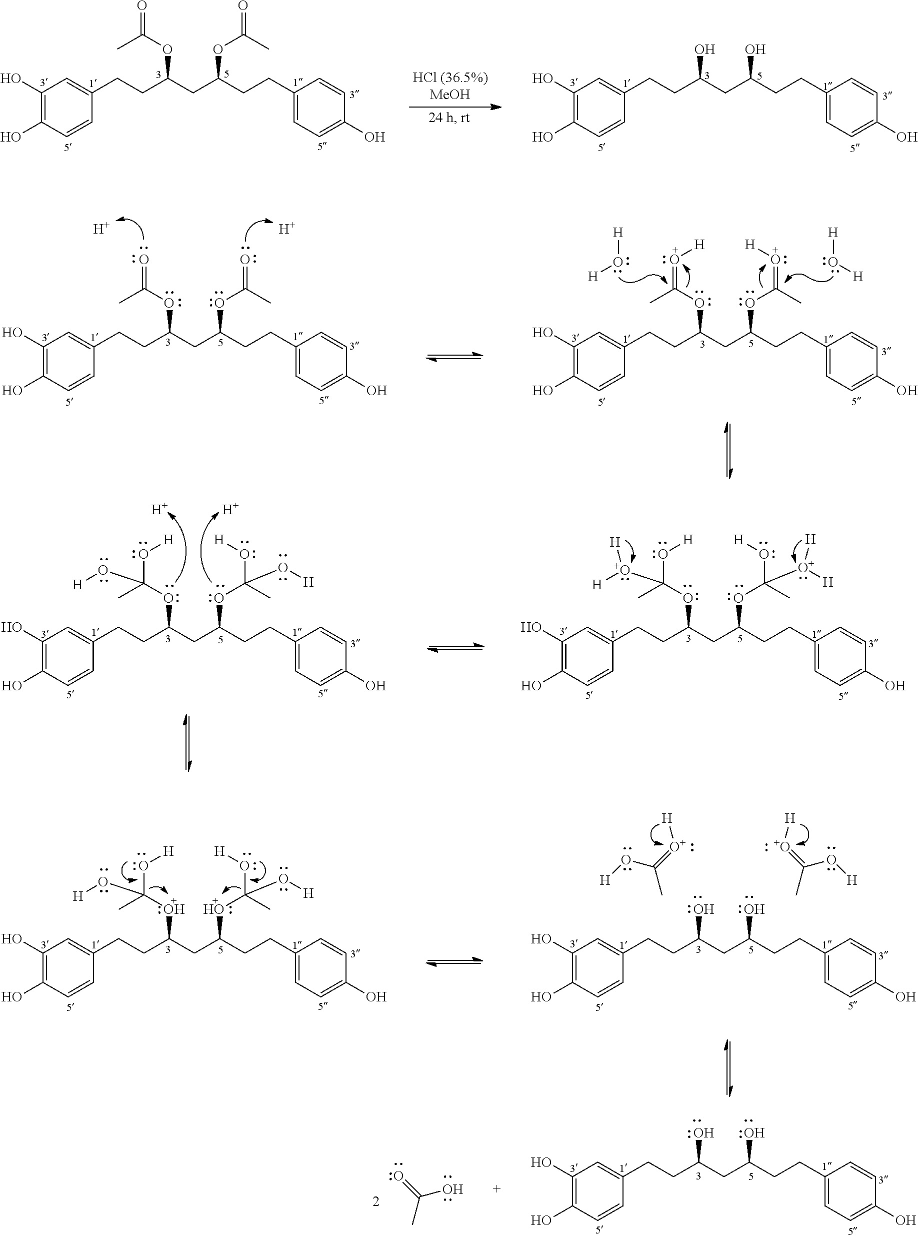

The effectiveness of the reaction is 72%.

Water is removed by eliminated by using sodium sulfate (Na₂SO₄). Methanol (MeOH) is also removed by centrifugation. Finally, the resultant compound is undergone column chromatography with CHCl₃:MeOH at volume or weight ratio (% w/w or % v/v) equaled to 95:5. The final result is compound 304 described by the following formula.

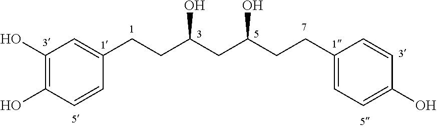 [SMILES: Oc1ccc(CC[C@H](O)C[C@H](O)CCc2ccc(O)c(O)c2)cc1]

The chemical properties of compound 304 will be described in FIG. 3-FIG. 5.

Next, referring to FIG. 3, a chemical formula 300 of the diarylheptanoid enantiomers extracted from white turmeric in accordance with exemplary aspects of the present invention are illustrated. Diarylheptanoid enantiomers 300 include a first aryl group 310, a linear heptane moiety 330 which is a 7 carbon linear chain, and a second aryl group 330, all are covalently bonded together. First aryl group 310 has a functional group R₂ at position 3, a hydroxyl (OH) functional group at position 4 and a second functional group R₃ at location 5. Similarly, second aryl group 330 has a functional group R₄ at position 3 and a hydroxyl function group (—OH) at position 5. Second aryl group 320 has two functional groups R₁ bonded at location 3 and location 5, C₃ and C₅. Depending on functional groups and their locations, diarylheptanoid compounds 300 produces different inhibitory urease activities. A compound 401 is obtained when R₁ functional groups are Acetocyl (OAc), R₂ functional group is hydroxyl, R₃ and R₄ functional groups are hydrogen (H). R₁ functional groups are in locations 3 and 5 are in syn-location relative to of heptane moiety 330. “Syn-” indicates that both functional groups are present on the same side of heptane 330. On the other hand, the “anti-” indicates that functional groups R₁ are on the opposite side of heptane moiety 330. A compound 402 is obtained when is obtained when R₁ functional groups are acetoxyl (OAc), R₂ functional group is Methoxy (OMe) while R₃ and R₄ are both hydrogen (H). In compound 402, R₁ functional groups are in locations 3 and 5 and are in syn-locations relative to linear heptane moiety 330. A compound 403 is obtained when is obtained when R₁ functional groups are hydroxyl (—OH), R₂ and R₃ functional groups are Methoxy (OMe), R₃ is also OMe, and R₄ are both hydrogen (H). In compound 403, R₁ functional groups are in locations 3 and 5 are in syn-locations relative to of heptane 330. A compound 304 is obtained when is obtained when R₁ and R₂ functional groups are hydroxyl (—OH) while R₃ and R₄ are both hydrogen (H). In compound 404, R₁ functional groups are in locations 3 and 5 which are in syn-locations relative to of linear heptane moiety 330. A compound 305 is obtained when is obtained when R₁ functional groups are hydroxyl (—OH), R₂ functional group is Methoxy (OMe), R₃ and R₄ are both hydrogen (H). In compound 305, R₁ functional groups are in locations 3 and 5 which are in syn-locations relative to of linear heptane moiety 330. A compound 306 is obtained when R₁ functional groups are hydroxyl (—OH), R₂ and R₃ functional group are Methoxy (OMe), R₄ are both hydrogen (H). In compound 306, R₁ functional groups are in locations 3 and 5 which are in syn-locations relative to linear heptane moiety 330. A compound 307 is obtained when R₁ functional groups are hydroxyl (—OH), R₂, R₃, and R₄ functional group are Methoxy (OMe). In compound 307, R₁ functional groups are in locations 3 and 5 which are in syn-locations relative to linear heptane moiety 330. A compound 308 is obtained when R₁ functional groups are hydroxyl (—OH), R₂, R₃, and R₄ functional group are Methoxy (OMe). In compound 308, R₁ functional groups in locations 3 and 5 are in anti-locations relative to linear heptane moiety 330. A compound 309 is obtained when R₁ functional groups are hydroxyl (—OH), R₂ functional group is Methoxy (OMe) while R₃ functional group is hydrogen (H) and R₄ functional group is hydroxyl (OH). In a compound 309, R₁ functional groups are in locations 3 and 5 which are in syn-locations relative to of linear heptane moiety 330.

Referring now to FIG. 4, chemical formulas 400 representing three new enantiomers 401-403 extracted from white turmeric rhizomes in accordance with an exemplary aspect of the present invention are shown. Refer to step 101 and step 201 above.

Compound 401,

$${[ α ]}_{D}^{25} - 30.5$$

(c 0.10, MeOH), is isolated as a yellow gel and found soluble in acetone. The molecular formula of compound 401 is determined as C₂₃H₂₈O₇ by high resolution electrospray ionization mass spectroscopy (HRESIMS) (m/z 415.4656 [M-H]⁻; calcd. m/z 415.4625). The infrared (IR) spectrum reveals the hydroxyl (3379 cm⁻¹), carbonyl (1703 cm⁻¹), and benzyl moiety groups (1612, 1514, and 1448 cm-1). The proton nuclear magnetic resonance (¹H-NMR) spectrum in the downfield region show the signal of one 1,3,4-trisubstituent benzene ring [δ_{H} 6.71 (1H, d, J=8.0 Hz, H-5′), 6.68 (1H, d, J=2.1 Hz, H-2′), and 6.51 (1H, dd, J=8.0 and 2.1 Hz, H-6′)] and a 1,4-disubstituent benzene ring [δ_{H} 7.01 (2H, d, J=8.5 Hz, H-2″ and H-6″) and 6.74 (2H, d, J=8.5 Hz, H-3″ and H-5″)]. The up field region presents the signals of two oxymethine groups [δ_{H} 4.96 (2H, ddt, J=12.7, 8.7, and 6.9 Hz, H-3 and H-5)], five methylene groups [δ_{H} 2.52 (2H, dt, J=13.3 and 7.7 Hz, H-1a and H-7a), 2.48 (2H, m, J=13.3 and 5.1 Hz, H-1b and H-7b), 1.86 (1H, dt, J=14.2 and 6.9 Hz, H-4a), 1.79 (1H, m, H-4b), 1.80 (4H, m, H-2 and H-6)] and two methyl groups [δ_{H} 1.96 (6H, s, 3-OCOCH₃ and 5-OCOCH₃)]. The carbon-13 nuclear magnetic resonance (¹³C-NMR) spectrum reveals the signals of 21 carbons, including two carbonyl carbons (6c 170.8), 12 aromatic carbons (6c 156.4, 148.8, 144.0, 134.2, 133.2, 116.2, 116.0, 115.9, 130.0, and 120.3). In the up field region, there are the signals of two oxymethine carbons (OCH) (δ_{C} 70.6 and 70.5), five methylene carbons (δ_{C} 39.1, 37.6, 31.5, and 31.3), and two methyl carbons (δ_{C} 21.1) (Table 1). The above 1D-NMR data indicate compound 401 to be a diarylheptanoid compound. The structure prediction of compound 401 is accomplished by analysis of the heteronuclear single quantum coherence spectroscopy (HSQC), correlation spectroscopy (COSY), and heteronuclear multiple bond correlation (HMBC) data. In particular, the 1H-1H-COSY signals of H₂-1/H₂-2/H-3/H₂-4/H-5/H2-6/H₂-7 indicate the appearance of a heptane chain moiety (linear heptane moiety 330). The signals of ABX-coupled three aromatic protons, combined with the HMBC correlations, allow the suggestion of a benzene ring A (first aryl 310). For ring B (second aryl 320), two pairs of ortho-coupled aromatic protons together with the heteronuclear multiple bond correlation (HMBC) keys of proton H-2″/H-6″ to carbon C-3″/C-5″ complete their assignments of the signals. The linkage of alkyl and aromatic groups was conducted by the HMBC correlations of H-1 to C-2′/C-6′ and of H-7 to C-2″/C-6″, indicating the connection of C-1-C-1′ and C-7-C-1″, respectively. The HMBC correlations from H-3 and H-5 to carbonyl carbon suggest two acetyl groups linked to carbon C-3 and C-5. The evidence shows that planar structure of compound 401 is assigned as 3,5-diacetoxy-1-(3′,4′-dihydroxyphenyl)-7-(4″-hydroxyphenyl)heptane. Deacetylation of compound 401 with HCl/MeOH yielded a 3,5-dihydroxyl derivative whose NMR data coincide with compound 304, rel-(3R,5S)-3,5-dihydroxy-1-(3′,4′-dihydroxyphenyl)-7-(4″-hydroxyphenyl)heptane. This fact suggests that the two acetyl groups in compound 401 are the syn configuration instead of the anti-conformation as a previously known compound, (3R,5R)-3,5-diacetoxy-1-(3′,4′-dihydroxyphenyl)-7-(4″)-hydroxyphenyl)heptane, isolated from Curcuma kwangsiensis³⁶. Therefore, compound 401 is identified as rel-(3R,5S)-3,5-diacetoxy-1-(3′,4′-dihydroxyphenyl)-7-(4″-hydroxyphenyl)heptane or aromatimin A for short. It is named as “aromatimin” due to its saturated C7-chained diarylheptanoid framework, predominantly found in C. aromatica species. Compound 401 is defined by the following formula:

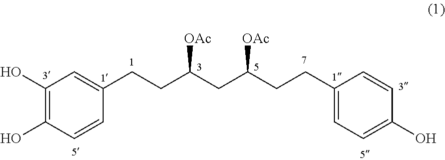 [SMILES: CC(=O)O[C@H](CCc1ccc(O)c(O)c1)C[C@H](CCc1ccc(O)cc1)OC(C)=O]

Where OAc is acetoxyl (CH₃COO—) and OH is hydroxyl group.

Compound 402,

$${[ α ]}_{D}^{25} = - 35.1$$

(c 0.10, MeOH), is obtained as a yellowish gel and found dissolved in chloroform. The molecular formula is determined as C₂₄H₃₀O₇ by high resolution electrospray ionization mass spectroscopy (HRESIMS) (m/z 429.1912 [M-H]⁻; calcd. m/z 429.1919). The infrared (IR) spectrum reveals the hydroxyl (3416 cm⁻¹), carbonyl (1731 and 1713 cm⁻¹), and benzyl moiety groups (1611, 1519, and 1451 cm⁻¹). A comprehensive analysis of the nuclear magnetic resonance (NMR) spectral data indicates a structural similarity between compounds 402 and 401. The key disparity between them is identified as the presence of a methoxyl group (OMe) at C-3′ in compound 402, whereas compound 401 contains a hydroxyl group at the same position (δ_{H} 3.86; δ_{C} 56.1) in compound 402 (please refer to Table 1). This deduction is substantiated by a heteronuclear multiple bond correlation (HMBC) correlation, which connects the proton of the methoxyl group (OMe) to carbon C-3′. Based on the accumulated evidence, the planar structure of compound 402 is assigned as 3,5-diacetoxy-1-(4-hydroxy-3-methoxyphenyl)-7-(4-hydroxyphenyl)heptane. This particular configuration was previously reported in the extract of Zingiber officinale Rosc, utilizing the ultra-high-performance liquid chromatography coupled to a quadrupole time-of-flight mass spectrometer (UHPLC-ESI-QTOF-MS/MS) method³⁷. To establish the stereochemistry at carbons C-3 and C-5, compound 402 undergoes deacetylation with HCl/MeOH, resulting in a derivative with NMR data closely resembling those of compound 405, namely rel-(3R,5S)-3,5-dihydroxy-1-(4-hydroxy-3-methoxyphenyl)-7-(4-hydroxyphenyl)heptane.

Consequently, the syn-configuration of the 3,5-dihydroxyl groups in compound 402 is unequivocally determined. Compound 402 corresponds to rel-(3R,5S)-3,5-diacetoxy-1-(4-hydroxy-3-methoxyphenyl)-7-(4-hydroxyphenyl)heptane or aromatimin B. Compound 402 is defined by the following formula:

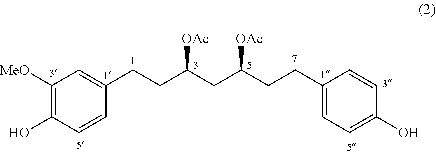 [SMILES: COc1cc(CC[C@H](C[C@H](CCc2ccc(O)cc2)OC(C)=O)OC(C)=O)ccc1O]

Where OAc is acetoxyl (CH₃COO—) and OH is hydroxyl group, and MeO is methoxy group (—OCH₃).

Compound 403,

$${[ α ]}_{D}^{25} = - 24. 5$$

(c 0.10, MeOH), was obtained as a yellowish gel and found dissolved in chloroform. The molecular formula is determined as C₂₅H₃₂O₈ by HRMSESI (m/z 459.2017 [M-H]⁻; calcd. 459.2024). The infrared (IR) spectrum reveals the hydroxyl (3433 cm⁻¹), carbonyl (1728 cm⁻¹), and benzyl moiety groups (1612, 1516, and 1458 cm⁻¹). A comprehensive analysis of the NMR spectral data reveals that structure of compound 403 is similar to compound 402. The primary difference observed is the presence of a methoxy group at C-5′ in compound 403, which replaces an aromatic proton present at the same position in compound 402 (see Table 1). This deduction is confirmed by a heteronuclear multiple bond correlation (HMBC) key from the proton methoxyl group to carbon C-5′. The planar structure of 403 is assigned as 3,5-diacetoxy-1-(4-hydroxy-3,5-dimethoxyphenyl)-7-(4-hydroxyphenyl)heptane. Relating the determination of stereochemistry, compound 403 is deacetylate with HCl/MeOH to yield a derivative whose NMR data are similar to those of compound 306, rel-(3R,5S)-3,5-dihydroxy-1-(4-hydroxy-3,5-dimethoxyphenyl)-7-(4-hydroxyphenyl)heptane. Thus, the syn-configuration of two hydroxyl groups is assigned for 3, which suggests rel-(3R,5S)-3,5-diacetoxy-1-(4-hydroxy-3,5-dimethoxyphenyl)-7-(4-hydroxyphenyl)heptane or aromatimin C. Compound 403 is defined by the following formula:

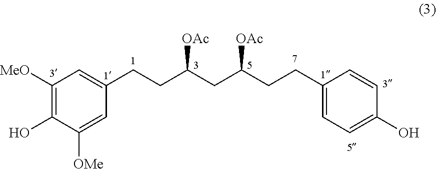 [SMILES: COc1cc(CC[C@H](C[C@H](CCc2ccc(O)cc2)OC(C)=O)OC(C)=O)cc(OC)c1O]

Where OAc is acetoxyl (CH₃COO—) and OH is hydroxyl group, and MeO is methoxy group (—OCH₃).

TABLE 1
The ¹H NMR (500 MHz) and ¹³C NMR (125 MHz) spectral
data of compounds 401-403

| | 401^{a} | | 402^{b} | | 403^{b} | |
|---|---|---|---|---|---|---|
| No. | δ_{H} J in Hz | δ_{C} | δ_{H} J in Hz | δ_{C} | δ_{H} J in Hz | δ_{C} |
| 1 | 2.52 dt | 31.3 | 2.56 dt (13.6, | 31.4 | 2.53 dt | 32.2 |
| | (13.3, 7.7) | | 7.7) | | (13.8, 8.8) | |
| | 2.48 dt | | 2.52 dt (13.6, | | 2.48 dt | |
| | (13.3, 5.1) | | 6.3) | | (13.8, 6.2) | |
| 2 | 1.80 m | 37.6 | 1.83 ddd | 36.1 | 1.84 m | 36.9 |
| | | | (11.8, 7.7, | | | |
| | | | 6.3) | | | |
| 3 | 4.96 ddt | 70.5 | 4.93 ddt | 71.0 | 4.93 ddt | 71.4 |
| | (12.7, 8.7, | | (11.8, 7.1, | | (13.0, 7.2, | |
| | 6.9) | | 5.8) | | 5.7) | |
| 4 | 1.86 dt | 39.1 | 1.95 dt (14.3, | 38.6 | 1.89 dt | 39.2 |
| | (14.2, 6.9) | | 7.1) | | (14.6, 7.2) | |
| | 1.79 m | | 1.76 dt (14.3, | | 1.83 m | |
| | | | 5.8) | | | |
| 5 | 4.96 ddt | 70.6 | 4.93 ddt | 71.0 | 4.93 ddt | 71.3 |
| | (12.7, 8.7, | | (11.8, 7.1, | | (13.0, 7.2, | |
| | 6.9) | | 5.8) | | 5.7) | |
| 6 | 1.80 m | 37.6 | 1.83 m | 36.0 | 1.84 m | 36.7 |
| 7 | 2.52 dt | 31.5 | 2.56 dt (13.6, | 30.8 | 2.53 dt | 31.2 |
| | (13.3, 7.7) | | 7.7) | | (13.8, 8.8) | |
| | 2.48 dt | | 2.52 dt (13.6, | | 2.48 dt | |
| | (13.3, 5.1) | | 6.3) | | (13.8, 6.2) | |
| 1′ | | 134.2 | | 133.3 | | 135.1 |
| 2′ | 6.68 d (2.1) | 116.2 | 6.65 d (1.9) | 111.2 | 6.47 s | 106.7 |
| 3′ | | 148.8 | | 146.6 | | 148.6 |
| 4′ | | 144.0 | | 144.0 | | 132.6 |
| 5′ | 6.71 d (8.0) | 115.9 | 6.81 d (8.0) | 114.5 | | 148.6 |
| 6′ | 6.51 dd (8.0, | 120.3 | 6.63 dd (8.0, | 121.1 | 6.47 s | 106.7 |
| | 2.1) | | 1.9) | | | |
| 1″ | | 133.2 | | 133.3 | | 133.1 |
| 2″/6″ | 7.01 d (8.5) | 130.0 | 6.99 d (8.4) | 129.5 | 6.99 d (8.5) | 130.6 |
| 3″/5″ | 6.74 d (8.5) | 116.0 | 6.73 d (8.4) | 115.4 | 7.74 d (8.5) | 116.0 |
| 4″ | | 156.4 | | 154.2 | | 156.4 |
| 3′-OMe | | | 3.86 s | 56.1 | 3.79 s | 56.6 |
| 5′-OMe | | | | | 3.79 s | 56.6 |
| 3-OAc | 1.96 s | 170.8 | 2.02 s | 171.0 | 1.99 s | 170.8 |
| | | 21.1 | | 21.2 | | 21.1 |
| 5-OAc | 1.96 s | 170.8 | 2.00 s | 171.0 | 1.96 s | 170.7 |
| | | 21.1 | | 21.3 | | 21.1 |

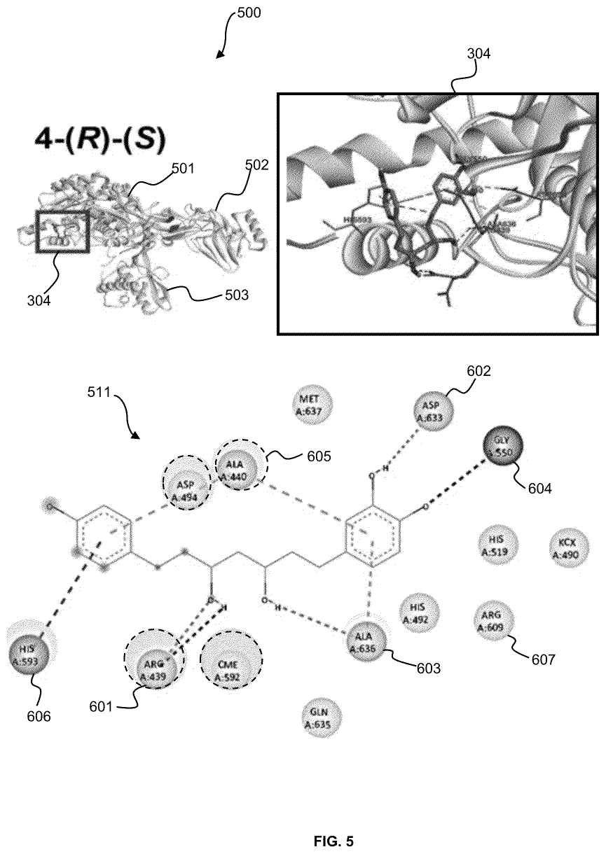

Referring now to FIG. 5-FIG. 8, respective diagrams 500-800 obtained from a structure activity relationship (SAR) software program illustrate binding poses and interactions of isomer 304 and 309 in the active sites of urease. FIG. 5-FIG. 8 also illustrate step 102 of method 100 and method 200. As described above, a three dimensional (3D) SAR model 500-800 provide relation between the chemical structures of the molecules 304, 309 and their respective therapeutic efficacies. In other words, SAR analysis provides justification about the most effective chemical (functional) group which triggers the therapeutic to the next level. Three dimensional (3D) SAR models 500-800 are capable of investigating stereochemistry and compounds that depend on chirality. A software program from StarDrop's Glowing Molecule™ is used to build 3D SAR models 511-514.

The urease inhibitory activities of the isolated compounds 401-403 and 304 to 309 are assessed, and the results were summarized in Table 2. For comparative purposes, hydroxyurea, a well-established urease inhibitor and a clinically available drug, is utilized as a positive control³⁸. The tested compounds exhibit concentration-dependent inhibitory effects on urease activity. Compounds 403, 307, and 308 demonstrate relatively weak anti-urease effects, while the remaining compounds exhibit substantial enzyme inhibition. Compounds 304 and 309 show the most potent inhibitory activity against urease (IC₅₀ values of 9.6 μM and 21.8 μM, respectively) higher than hydroxyurea (77.4 μM).

TABLE 2
Urease inhibitory activities and binding
affinity of nine diarylheptanoids

| | | | Binding affinity (kcal/mol) | | | |
|---|---|---|---|---|---|---|
| Comp. | IC₅₀ (μM) | | 3R,5R | 3R,5S | 3S,5R | 3S,5S |
| 401 | 129.3 | | | −6.65 | −6.53 | |
| 402 | 223.1 | | | −6.01 | −6.40 | |
| 303 | >250 | (3.3**) | | −5.66 | −5.70 | |
| 304 | 9.6 | | | −6.90 | −6.88 | |
| 305 | 152.7 | | | −6.23 | −6.15 | |
| 306 | 228.3 | | | −6.14 | −6.12 | |
| 307 | >250 | (27.3**) | | −5.89 | −5.70 | |
| 3088 | >250 | (14.3**) | −5.88 | | | −5.70 |

| | | | |
|---|---|---|---|
| 309 | 21.8 | −6.96 | −6.45 |

| | | |
|---|---|---|
| PC* | 77.5 | −4.58 |

Based on the abovementioned findings and previously collected data³¹, a comprehensive understanding of the structure-activity relationship (SAR) of diarylheptanoids 300 as described in FIG. 3 is established. According to the stereo chemical configuration of these compounds, the conformational arrangement of stereoisomers does not significantly influence the inhibitory activity of urease enzymes by linear diarylheptanoids. The syn-configuration exhibits a similar inhibitory efficacy compared to the anti-configuration (307-308). Substituting the 3,5-diacetyl groups with the 3,5-dihydroxyl groups along the linear heptane chain leads to a pronounced surge in activity. Inhibitory activity of urease enzymes of compounds 401-403 and 304-309 are defined respectively as IC₅₀401, IC₅₀402, IC₅₀403, IC₅₀304, IC₅₀305, IC₅₀306, IC₅₀307, IC₅₀308, IC₅₀309 (IC₅₀304>>IC₅₀401, IC₅₀305>IC₅₀402, IC₅₀306>IC₅₀403, and (−)-hannokinol>(3R,5R)-3,5-diacetoxy-1,7-bis(4-hydroxyphenyl)heptane). Where IC₅₀ is defined as half-maximal inhibitory concentration. IC₅₀ is used to measure drug potency of a compound. Within the meaning of the present invention, enhanced urea enzyme inhibitory activity has the same meaning as the compound which has the highest IC₅₀ or the highest potency. Within benzene ring A or first aryl group 310, the addition of hydroxyl (OH) or methoxyl (OMe) group at the C-3′ position significantly enhances the anti-urease activity (IC₅₀305>(−)-hannokinol, IC₅₀402>(3R,5R)-3,5-diacetoxy-1,7-bis(4-hydroxyphenyl)heptane, IC₅₀304>(−)-hannokinol, and IC₅₀401>(3R,5R)-3,5-diacetoxy-1,7-bis(4-hydroxyphenyl)heptane). Moreover, the inclusion of a hydroxyl group at the C-3′ position wielded a more potent anti-urease effect than a methoxyl group (IC₅₀401>IC₅₀402, IC₅₀304>>IC₅₀405). The introduction of an additional methoxyl group at C-5′ induced a substantial reduction in activity (IC₅₀403<IC₅₀402, IC₅₀306<IC₅₀305). In some cases, an outright loss of urease inhibitory potential emerged when the 4-hydroxyphenyl ring (B) hosted an extra methoxyl group (OMe) at C-3″ (IC307 or IC₅₀308<IC₅₀306). The hydroxylation at the C-3″ group emerged as a pivotal factor in ushering urease inhibition (IC₅₀309>>IC₅₀305). These analyses underscore the paramount role played by hydroxyl groups (OH) in heightening the activity against urease. Conversely, methylation of benzene rings or acetylation of linear heptane moiety 330 is observed to curtail the activity unfavorably.

Continuing with FIG. 5-FIG. 8, in docking simulations, the root-mean-square deviation (RMSD) of 0.645 Å between the docked pose and the experimental structure of the co-crystallized ligand is investigated. This demonstrates that the docking models 511-514 proficiently generate ligand poses closely resembling those encountered in experimentations. The molecular docking result is evaluated based on the binding affinity value and the pattern of bonding illustrated through a pharmacophore diagram. After docking, the ligands were ranked in descending order based on their binding affinities. The docked pose with the most negative binding energy value has the highest affinity and is the best-docked conformation. In molecular docking, binding affinity is influenced by non-covalent intermolecular interactions such as hydrogen bonding, electrostatic interactions, hydrophobic interactions, and Van der Waals forces. Before analyzing the docking result, it is noticed from the crystal structure of urease in complex with acetohydroxamic acid (AHA—a standard urea inhibitor) that AHA interacts with the active site of urease by forming two hydrogen bonds with the carbamylated Lys490 and Asp633 602. The docking results reveal that the control hydroxyurea displays a binding pose with a binding affinity score of −4.58 kcal/mol and almost overlaps with AHA by forming two hydrogen bonds formed with Lys490 and Asp633. This result agrees with previous study about the substrate-competitive binding mechanism of these two known inhibitors²⁷.

As illustrated in Table 2, all the docked compounds exhibit better binding affinities than the positive control hydroxyurea. Specifically, compounds 304 and 309, in these two enantiomers, demonstrate the strongest binding affinities to urease compared to the control and the other compounds. In contrast, compounds 403, 307, and 308 show the weakest binding affinities. Interestingly, a strong correlation is observed between the docking binding affinities of the considered compounds and their corresponding inhibitory potencies.

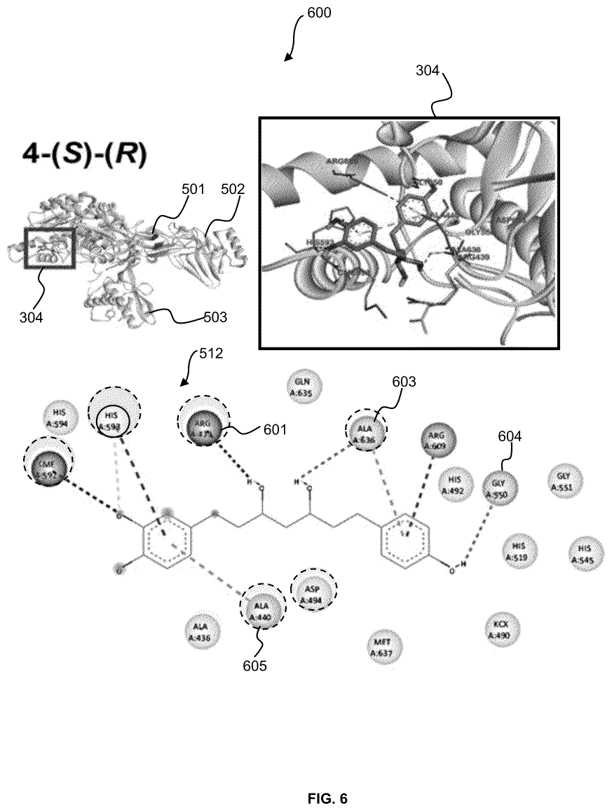

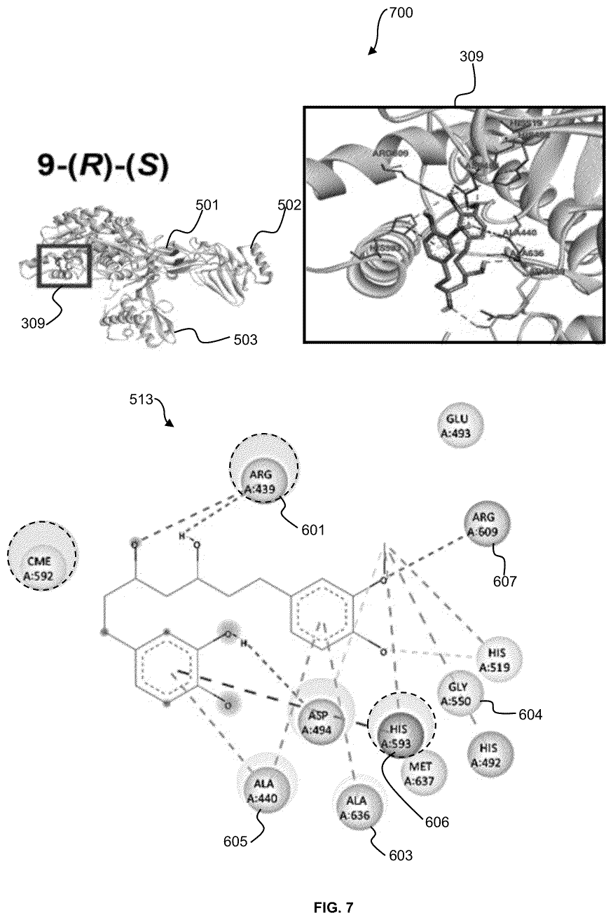

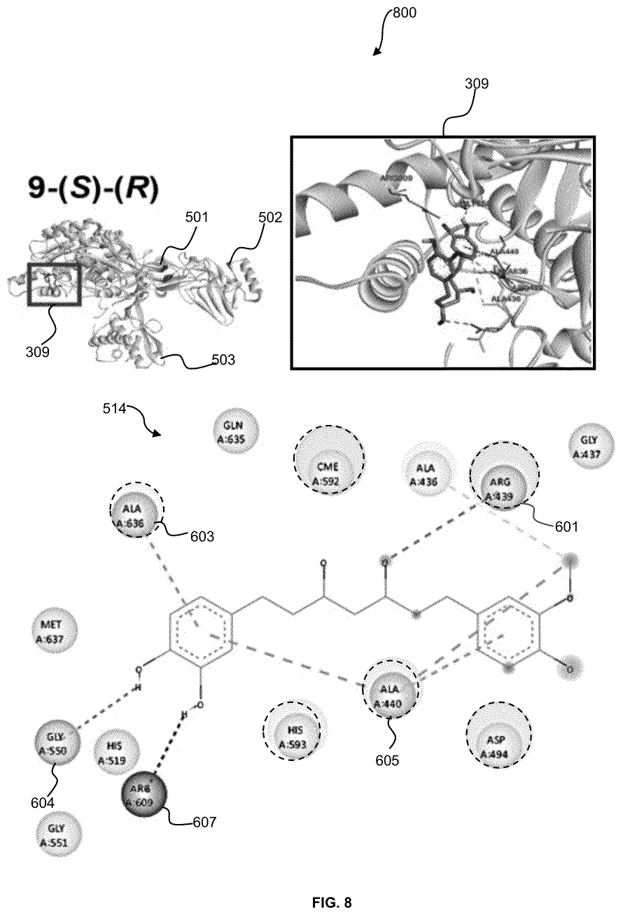

The docking models 511 in FIG. 5, 512 in FIG. 6, 513 in FIG. 7, and 514 in FIG. 8 also agree with the experiment test that substituting 3,5-diacetyl groups with 3,5-dihydroxyl groups along linear heptane moiety 330 (heptanoid chain) leads to a remarkable increase in binding affinity against the target (IC₅₀304>IC₅₀401, IC₅₀305>IC₅₀402, and IC₅₀306>IC₅₀403). Similarly, within first aryl moiety 310 (benzene ring A), in compounds with the presence of a hydroxylphenyl group at the C-3′ position rather than a methoxy group (OMe) at the same position, the docking scores of those compounds are observed to be better (IC₅₀401>IC₅₀402, IC₅₀304>IC₅₀305). In addition, when adding one more extra methoxy group at C-5′ into first aryl moiety 310 (benzene ring A), the binding affinity is observed to decrease substantially, as seen in the case of compound 403 versus 402 (IC₅₀403<IC₅₀402) and compound 306 versus 305 (IC₅₀306<IC₅₀305). Within second aryl moiety 320 (benzene ring B), the same trend of reduction in binding affinity was observed for compounds 307 and 308 compared to compound 306 (IC₅₀307, IC₅₀308<IC₅₀306). On the other hand, when an extra hydroxyl group was added at the C-3″ position in second aryl moiety 320 (ring B), the binding affinity was improved again (IC₅₀309>IC₅₀305). Besides demonstrating the favoring of hydroxyl groups in the structure of diarylheptanoid compounds by the enzyme urease, the docking experiment also supported the experimental result that there is no significant difference between the inhibitory activity of two stereoisomers of a compound. This observation is obtained when comparing the binding affinities of compounds 307 and 308 stereoisomers.

Continuing with FIG. 5-FIG. 8, the inhibitory mechanism of diarylheptanoid compounds, pharmacophore diagrams 500 of the best-docked diarylheptanoid compounds against urease (304 and 309) are analyzed in detail to learn about the structure-activity relationship (SAR) based on molecular interactions at the binding site 501. In general, binding site 501 of these compounds was shifted toward entrance flaps 502 and 503 compared to the binding site of the control hydroxyurea. In detail, the stereoisomers of compound 304 in FIG. 5-FIG. 6 are found to interact with urease by forming hydrogen bonds with Arg439 601, Asp633 602, Ala636 603, and Gly550 604. Hydrophobic interactions with Ala440 605, His593 606, and Arg609 607 also contributed to the binding affinity with the protein. Similarly, compound 309 in FIG. 7-FIG. 8 has most of the same critical pharmacophore characteristics as 304. Hydrogen bond interactions with residue Arg439 601 and the active site residue Arg609 607 were also observed in the pharmacophore of compound 309. The binding pattern of these two compounds suggests that they interact with residues lining the mobile flap and the active site of urease, blocking the entrance flapping region 502-503 and preventing the substrate urea 501 from entering the urease active site to undergo a hydrolysis reaction. The pharmacophore result also suggests that the bulky scaffold of diarylheptanoid and the presence of the hydroxyl groups can explain the superior inhibitory activity of diarylheptanoid compounds compared to known inhibitors.

In summary, the realization of step 102 and step 204, the extraction and isolation of C. aromatica rhizomes, results in three new diarylheptanoids 401-403 alongside six known diarylheptanoids 304-309. Biological activity testing of the compounds demonstrates moderate to strong inhibitory activity on urease. The structure and activity relationship (SAR) drawn from both inhibition assay and molecular docking reveals that the presence of hydroxyl groups leads to the superior inhibitory activity of the diarylheptanoid compounds. Besides, by comparing the relative position of the binding site of hydroxyurea and diarylheptanoid compounds, the mode of action of diarylheptanoid compounds 300 is proposed to be different from that of hydroxyurea. These compounds are predicted to bind to the “flapping” region 502-503 near the dinickel active center, causing the loss of catalytic activity.

Within the scope of the present invention, the potential of C. aromatica in the treatment of gastrointestinal illnesses through the ability to inhibit the urease activity of diarylheptanoids. Compounds exhibiting potent anti-urease activity are developed into novel pharmaceutical products. Moreover, information about the structure-activity relationship (SAR) and molecular insights from the computational model assist in the design of therapeutics against gastrointestinal illnesses. The mechanism of action of diarylheptanoids in the present invention is achieved by using a rigid protein model.

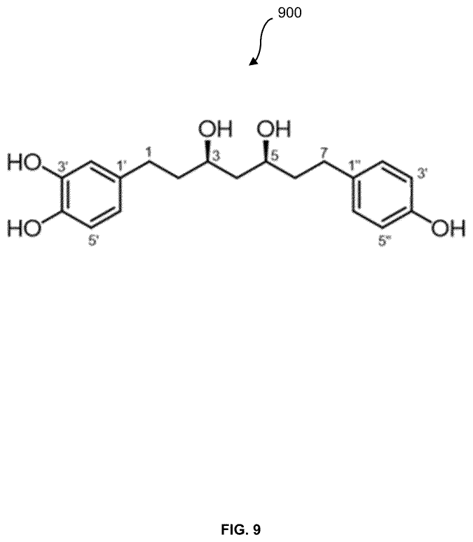

Finally, FIG. 9 illustrates chemical formulas for isomers 304 that is used in the treatment of gastric ulcers in accordance with an aspect of the present invention.

### EXPERIMENTS AND RESULTS

Various experiments were performed to realize the methods of the present invention. Optical rotations were measured using an A.KRÜSS Optronic P8000 polarimeter. Infrared (IR) spectra were recorded using a JASCO FT/IR-6600 spectrometer manufactured by JASCO International Co., Ltd. Ultraviolet (UV) spectra were obtained using a Shimadzu UV-1800 spectrophotometer from Shimadzu Pte., Ltd. Nuclear Magnetic Resonance (NMR) spectra were recorded on a Bruker Avance III 500 spectrometer by Bruker BioSpin AG, with deuterated solvents serving as internal standards. The chemical shifts in the NMR spectra are expressed in b values. High-resolution electrospray ionization mass spectrometry (HRESIMS) was performed using a Bruker micrOTOF QII spectrometer provided by Bruker Singapore Pte., Ltd. For analytical, column chromatography and preparative thin-layer chromatography (PTLC), silica gel 60, ODS silica gel, Kieselgel 60F254 or RP-18F254 plates, hydroxyurea, urea, and DMSO from Merck were used. The enzyme urease (EC 3.5.1.5) from Canavalia ensiformis (Jack bean) was obtained from Sigma-Aldrich Pte. Ltd. All other chemicals used were of the highest available grade.

### Plant Materials

The rhizomes of C. aromatica were harvested in March 2017 from Tinh Bien district, An Giang province, Vietnam. Authentication was performed by the National Institute of Medical Material. The corresponding voucher samples (DMC-9006) were securely stored at the Faculty of Chemistry, VNUHCM-University of Science, Vietnam.

### Extraction and Isolation

The desiccated rhizomes of C. aromatica (6.5 kg) were subjected to extraction using a Soxhlet extractor with methanol as the solvent. The resulting extract (834.9 g) was then suspended in water and successfully partitioned with n-hexane to eliminate fatty compounds, followed by chloroform to obtain the chloroform (CHCl₃) (272.2 g) extract. The CHCl₃-soluble extract underwent further refinement through silica gel column chromatography, employing acetone-CHCl₃ gradient mixtures (v/v, 0-100%) as eluents, isolating seventeen fractions, denoted as fr.A-fr.Q.³¹ In the present invention, fraction K (59.9 g) was passed over a CC (silica gel, Me₂CO-n-hexane) to yield eight sub-fractions (fr.K1, 135 mg; fr.K2, 69.3 mg; fr.K3, 386 mg; fr.K4, 137 mg; fr.K5, 174 mg; fr.K6, 22.4 g; fr.K7, 9.6 g; fr.K8, 6.1 g). Sub-fraction fr.K1 was purified by chromatography CC (silica gel, EtOAc-CHCl₃) to yield 6 (5.2 mg) and 8 (4.8 mg). Sub-fractions fr.K2 were chromatographed using CC (silica gel, CHCl₃-n-hexane, gradient 0-100% CHCl₃), and further purified by normal-phase preparative thin-layer chromatography (PTLC) (MeOH-IPA-CHCl₃-n-hexane mixture, 4:6:40:50, v/v/v/v) to afford 5 (10.3 mg). Sub-fraction fr.K3 was subjected to CC (ODS silica gel, MeOH—H₂O, gradient 5-80% MeOH) to obtain 3 (114.0 mg). Sub-fraction K4 was subjected using CC (silica gel, EtOAc-CHCl₃, gradient 0-80% EtOAc), followed by normal-phase PTLC (EtOAc-CH₂Cl₂-n-hexane mixture, 40:30:30, v/v) to give 7 (10.3 mg) and 2 (16.9 mg). Sub-fraction fr.K5 was applied using CC (silica gel, EtOAc-CHCl₃, gradient 0-90% EtOAc), and then purified by reserved-phase PTLC (H₂O-MeCN-MeOH mixture, 2:4:4, v/v/v) to furnish 4 (3.5 mg) and 9 (3.8 mg). Fraction fr.K6 was subjected by CC (silica gel, EtOAc-n-hexane mixture, gradient 5-100% EtOAc, v/v) to obtain compound 401 (15.3 g).

Aromatimin A (compound 401) Yellowish gel;

$${[ α ]}_{D}^{25} = - 30. 5$$

(c 0.10, MeOH), IR νₘₐₓ (KBr) 3379, 1703, 1612, 1514, and 1448 cm⁻¹; ¹H- (500 MHz, CD₃COCD₃) and ¹³C-NMR (125 MHz, CD₃COCD₃), see Table 1; HRESIMS m/z 415.4656 [M-H]⁻ (calcd. for C₂₃H₂₈O₇, 415.4625).

Aromatimin B (compound 402) Yellowish gel;

$${[ α ]}_{D}^{25} = - 35. 1$$

(c 0.10, MeOH), IR νₘₐₓ (KBr) (3416, 1731, 1713, 1611, 1519, and 1451 cm⁻¹; ¹H- (500 MHz, CHCl₃) and ¹³C-NMR (125 MHz, CD₃COCD₃), see Table 1; HRESIMS m/z 429.1912 [M-H]⁻ (calcd. for C₂₄H₃₀O₇, 429.1919).

Aromatimin C (compound 403) Yellowish gel;

$${[ α ]}_{D}^{25} = - 24. 5$$

(c 0.10, MeOH), IR νₘₐₓ (KBr) (3433, 1728, 1612, 1516, and 1458 cm⁻¹; ¹H- (500 MHz, CD₃COCD₃) and ¹³C-NMR (125 MHz, CD₃COCD₃), see Table 1; HRESIMS m/z 459.2017 [M-H]⁻ (calcd. for C₂₅H₃₂O₈, 459.2024).

### Unrease Inhibition Assay

Previously reported was the inhibitory activity of urease¹⁹, with slight modifications made in the concentrations ranging from 250 μM to 1 μM. The reaction was initiated by adding 60 mM urea (500 μL) to a 30 U/mL urease solution (50 μL) in 0.01 M phosphate buffer pH 7.0 (920 μL) and incubating at room temperature for 30 min. Following this, the mixtures were further incubated for 20 min. Next, 1 mg/mL red phenol reagent (30 μL) was added and incubated for 10 min to detect the reaction. The anti-α-urease activity of the samples was determined by measuring the decrease in absorbance at 556 nm. A positive control, hydroxyurea, was utilized in this screening.

The inhibitory percentage of the samples was calculated using the following equation:

$$I \% = \frac{{A}_{0} - {A}_{1}}{{A}_{0}} \times 100 \% .$$

where I is inhibition, A₀ and A₁ represent the activities of the enzyme without and with the test sample, respectively. The enzyme inhibitory percentage was used to determine the activity of the samples in the above assay. IC₅₀ values were determined from the mean data obtained from these experiments for the tested activities.

### Electronic Structure and Calculations and Molecular Docking

The molecular docking experiments were carried out using AutoDock Vina (version 1.2.3)³⁹, one of the fastest and most widely used open-source programs for molecular docking, to predict the binding affinity and molecular interactions of ligands on the target protein receptor. The 3D atomic structures of 7 diarylheptanoids were built and geometrically optimized using B3LYP/6-31G(d, p) level of theory implemented in the Gaussian 16 package⁴⁰ and eventually saved as PDB format. Each diarylheptanoid compound has two chiral centers at C-3 and C-5, resulting in four enantiomers. To perform molecular docking, the 3D structure of the Canavalia ensiformis urease was retrieved from the first crystallized structure of jack bean urease in complex with AHA obtained by X-ray diffraction at 1.52 Å, which was published on the RCSB PDBn (Research Collaboratory for Structure Bioinformatics Protein Data Bank entry 4H9M). The coordinates of AHA were used to locate the center of the docking grid box, which sets the docking boundary. In this experiment, we set a grid box with dimensions of 25×25×25 (A) with grid box resolution 1 Å (illustrated in FIG. S1.A), the searching exhaustiveness of 1000. To validate the reliability of the docking simulations, the crystal structure of urease with its co-crystallized ligand (AHA as in this case) was re-docked. The root mean square deviation (RMSD) values between the docked and the original conformation of the known inhibitor AHA in the complex were calculated. If the RMSD of the docked pose was less than 2.5 Å from the experimentally observed conformation, the docking experiment was considered to be successful⁴¹. The PDB files of the protein and the ligands were converted to the PDBQT Autodock structure format and ready to be used for docking with Autodock Vina. During the conversion step, Gasteiger charges were added for the ligands, and all rotatable bonds were treated flexibly by Autodock tools (ADT) 1.5.7.⁴² All water, ions, and ligands cocrystallized in the complex were deleted for the protein. After that, only polar hydrogens and an equally spread Kollman charge were added to the protein. As rigid docking was used in this experiment, the protein structure was treated as a rigid body, whereas the ligands were allowed to be flexible. The docking results were ranked from the best to the worst according to their docking energy scores. The more negative the energy score, the better the rank of the binding mode. For each compound, 50 possible binding modes were generated; the chosen pose had the best binding score and was located in a binding cluster. Besides the binding affinity calculated from AutoDock Vina, a pharmacophore, which is an abstract description of the interactions of the docked compound at the binding site, was generated by BIOVIA Discovery Studio Visualizer⁴³

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should, therefore, be construed in accordance with the appended claims and any equivalents thereof.

### Abbreviation

δ_{H}=¹H-NMR chemical shift
δ_{C}=¹³C-NMR chemical shift
AHA=Acetohydroxamic acid is a standard urea inhibitor
API=Active pharmaceutical ingredient
BRS=Broad singlet peak
CC=Column chromatography
CD₃COCD₃=Deuterated acetone
COSY=Correlation Spectroscopy
d=Doublet peak
dd=Doublet of doublet peak
EtOAc=Ethyl acetate
HMBC=Heteronuclear Multiple Bond Correlation
HSQC=Heteronuclear Single Quantum Correlation
HR-ESI-MS=High resolution, Electron-sprayed Ionization Mass Spectrometry
GOLD=Genetic Optimization for Ligand Docking
I %=Inhibitory percentage
IC₅₀=Half maximal inhibitory concentration
J=Coupling constant
m=Multiplet peak
MeCN=Acetonitrile
MeOH=methanol (CH₃OH)
NMR=Nuclear magnetic resonance
OAc=Acetocyl
ODS=Octadecylsilyl-Silica
OMe=Methoxy
PTLC=Preparative thin layer chromatography
PDBQT=Partial Data Bank Partial Charge and Atom Type
RCSB PDB=Research Collaboratory for Structure Bioinformatics Protein Data Bank
RP: Reversed phase
s=Singlet peak
SAR=Structure Activity Relationship
t=Triplet peak
TOF=Time of flight

### REFERENCES

1. J. Labenz and G. Börsch, Gut, 1994, 35, 19.
2. H. Mobley, M. J. Cortesia, L. E. Rosenthal and B. D. Jones, J. Clin. Microbiol., 1988, 26, 831-836.
3. D. Y. Graham, J. Gastroenterol., 2000, 35, 90-97.
4. C. Follmer, Journal of clinical pathology, 2010, 63, 424-430.
5. J. B. Sumner, in A Source Book in Chemistry, 1900-1950, ed. H. M. Leicester, Harvard University Press, 1968, DOI: doi:10.4159/harvard.9780674366701.c115, pp. 322-327.
6. N. E. Dixon, C. Gazzola, R. L. Blakeley and B. Zerner, J. Am. Chem. Soc., 1975, 97, 4131-4133.
7. L. Mazzei, F. Musiani and S. Ciurli, in The Biological Chemistry of Nickel, eds. D. Zamble, M. Rowińska-Żyrek and H. Kozlowski, The Royal Society of Chemistry, 2017, DOI: 10.1039/9781788010580-00060, p. 0.
8. K. Kappaun, A. R. Piovesan, C. R. Carlini and R. Ligabue-Braun, J. Adv. Res., 2018, 13, 3-17.
9. M. J. Maroney and S. Ciurli, Chem. Rev., 2014, 114, 4206-4228.
10. B. Zambelli, L. Mazzei and S. Ciurli, in Progress in Molecular Biology and Translational Science, ed. V. N. Uversky, Academic Press, 2020, vol. 174, pp. 307-330.
11. W. N. Fishbein and P. P. Carbone, J. Biol. Chem., 1965, 240, 2407-2414.
12. W. N. Fishbein, T. S. Winter and J. Davidson, J. Biol. Chem., 1965, 240, 2402-2406.
13. N. E. Dixon, C. Gazzola, J. J. Watters, R. L. Blakeley and B. Zerner, J. Am. Chem. Soc., 1975, 97, 4130-4131.
14. D. P. Griffith, J. Gibson, C. Clinton and D. M. Musher, J. Urol., 1978, 119, 9-15.
15. A. J. Stemmler, J. W. Kampf, M. L. Kirk and V. L. Pecoraro, J. Am. Chem. Soc., 1995, 117, 6368-6369.
16. S. Benini, W. R. Rypniewski, K. S. Wilson, S. Miletti, S. Ciurli and S. Mangani, Structure, 1999, 7, 205-216.
17. S. Benini, W. R. Rypniewski, K. S. Wilson, S. Miletti, S. Ciurli and S. Mangani, J. Biol. Inorg. Chem., 2000, 5, 110-118.
18. M. Kot, W. Zaborska and K. Orlinska, J. Enzyme Inhib., 2001, 16, 507-516.
19. T. Tanaka, M. Kawase and S. Tani, Bioorg. Med. Chem., 2004, 12, 501-505.
20. W. Chen, Y. Li, Y. Cui, X. Zhang, H.-L. Zhu and Q. Zeng, Eur. J. Med. Chem., 2010, 45, 4473-4478.
21. Y. M. Cui, Y. Li, Y. J. Cai, W. Chen and H. L. Zhu, J. Coord. Chem., 2011, 64, 610-616.
22. L. Mazzei, M. Cianci, S. Benini and S. Ciurli, Angew. Chem. Int. Ed. Engl., 2019, 58, 7415-7419.
23. S. Svane, J. J. Sigurdarson, F. Finkenwirth, T. Eitinger and H. Karring, Sci. Rep., 2020, 10, 8503.
24. S. Milo, R. A. Heylen, J. Glancy, G. T. Williams, B. L. Patenall, H. J. Hathaway, N. T. Thet, S. L. Allinson, M. Laabei and A. T. A. Jenkins, Sci. Rep., 2021, 11, 3726.
25. E. S. Moghadam, A. M. Al-Sadi, M. Talebi, M. Amanlou, M. Shongwe, M. Amini and R. Abdel-Jalil, Lett. Drug. Des. Discov., 2022, 19, 1111-1120.
26. K. Pedrood, H. Azizian, M. N. Montazer, A. Moazzam, M. Asadi, H. Montazeri, M. Biglar, M. Zamani, B. Larijani and K. Zomorodian, Sci. Rep., 2022, 12, 13827.
27. S. Suenaga, Y. Takano and T. Saito, Molecules, 2023, 28, 2697.
28. F. Lodato, F. Azzaroli, L. Turco, N. Mazzella, F. Buonfiglioli, M. Zoli and G. Mazzella, Best Pract. Res. Clin. Gastroenterol., 2010, 24, 193-201.
29. N. M. Umar, T. Parumasivam, N. Aminu and S.-M. Toh, J. Appl. Pharm. Sci., 2020, 10, 180-194.
30. Á. Alberti, E. Riethmüller and S. Béni, J. Pharm. Biomed. Anal., 2018, 147, 13-34.
31. T. H. Le, T. Q. Bui, P. H. Dang, H. X. Nguyen, T. N. V. Do, M. T. T. Nguyen and N. T. Nguyen, Vietnam J. Chem., 2023, 62.
32. J. Li, C.-R. Liao, J.-Q. Wei, L.-X. Chen, F. Zhao and F. Qiu, Bioorganic Med. Chem. Lett., 2011, 21, 5363-5369.
33. R. G. Fuentes, K. Toume, M. A. Arai, T. Koyano, T. Kowithayakorn and M. Ishibashi, Heterocycles, 2014, 8, 1501-1509.
34. J. Ma, X. Jin, L. Yang and Z.-L. Liu, Phytochem. Lett., 2004, 65, 1137-1143.
35. K. Akiyama, H. Kikuzaki, T. Aoki, A. Okuda, N. H. Lajis and N. Nakatani, J. Nat. Prod., 2006, 69, 1637-1640.
36. S.-D. Chen, J.-T. Gao, J.-G. Liu, B. Liu, R.-Z. Zhao and C.-J. Lu, Fitoterapia, 2015, 102, 67-73.
37. F. Lu, H. Cai, S. Li, W. Xie and R. Sun, Molecules, 2022, 27, 7818.
38. A. Martelli, P. Buli and V. Cortecchia, Eur Urol., 1981, 7, 431-439.
39. J. Eberhardt, D. Santos-Martins, A. F. Tillack and S. Forli, J. Chem. Inf. Model, 2021, 61, 3891-3898.
40. M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, X. Li, M. Caricato, A. V. Marenich, J. Bloino, B. G. Janesko, R. Gomperts, B. Mennucci, H. P. Hratchian, J. V. Ortiz, A. F. Izmaylov, J. L. Sonnenberg, D. Williams-Young, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y. Honda, O. Kitao, H. Nakai, T. Vreven, K. Throssell, J. A. Montgomery Jr., J. E. Peralta, F. Ogliaro, M. J. Bearpark, J. J. Heyd, E. N. Brothers, K. N. Kudin, V. N. Staroverov, T. A. Keith, R. Kobayashi, J. Normand, K. Raghavachari, A. P. Rendell, J. C. Burant, S. S. lyengar, J. Tomasi, M. Cossi, J. M. Millam, M. Klene, C. Adamo, R. Cammi, J. W. Ochterski, R. L. Martin, K. Morokuma, O. Farkas, J. B. Foresman and D. J. Fox, Gaussian 16 Rev. C.01, Gaussian: Wallingford, CT, 2016.
41. J. Dickerhoff, K. R. Warnecke, K. Wang, N. Deng and D. Yang, Int J Mol Sci., 2021, 22, 10801.
42. G. M. Morris, R. Huey, W. Lindstrom, M. F. Sanner, R. K. Belew, D. S. Goodsell and A. J. Olson, Journal of computational chemistry, 2009, 30, 2785-2791.
43. 13, 2016.

1. A composition, comprising:
a diarylheptanoid compound, having a first aryl moiety covalently bonded to a linear heptane moiety and to a second aryl moiety, described by the following formula: 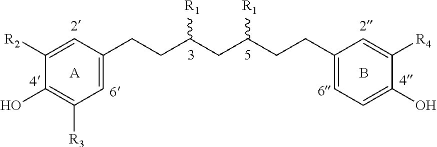
Wherein R₁ is a first functional groups at a location 3,5 of said linear heptane moiety,
R₂ is a second functional group at a location 3 of said first aryl group,
R₃ is a third functional group at a location 5 of said first aryl group,
R₄ is a fourth function group at a third location of said second aryl group, and
OH is a hydroxyl functional group at a fifth location of said second aryl group.

2. The composition of claim 1, wherein said compound is extracted and isolated from a white turmeric (Curcuma aromatica Salisb.).

3. The composition of claim 2 wherein said first functional group R₁ is a hydroxyl group, said second functional group R₂ is a hydroxyl group (OH), said third functional group R₃ and said fourth functional group R₄ are hydrogen (H), which is defined by the following formula:

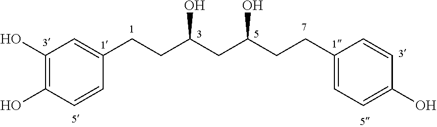 [SMILES: Oc1ccc(CC[C@H](O)C[C@H](O)CCc2ccc(O)c(O)c2)cc1]

4. The composition of claim 3 wherein said first functional groups R₁ at location 3,5 chain are on the same side (-syn) of said linear heptane moiety.

5. The composition of claim 4 characterized by a half maximal inhibitory concentration (IC₅₀) of 9.6 μM against urease enzyme.

6. The composition of claim 5 characterized by a binding affinity of −6.88 kcal/mol with said urease enzyme.

7. A method for synthesizing a diarylheptanoid composition characterized by a inhibitory urease activity, comprising:
(a) preparing white turmeric rhizomes (C. aromatic);
(b) isolating and extracting said diarylheptanoid compound from said white turmeric rhizomes to obtain a plurality of sub-fractions;
(c) modeling and optimizing said plurality of sub-fractions using a structure activity relationship (SAR), a half maximal inhibitory concentration (IC₅₀), molecular docking, and electronic structure calculations to select a precursor sub-fraction and to design an optimal sub-fraction that have the highest IC₅₀ among said sub-fractions; and
(d) performing chemical reactions of said precursor sub-fraction to obtain said plurality of sub-fractions which are defined by the formula: 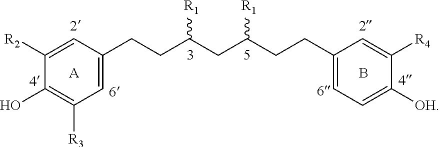
wherein R₁ is a first functional groups at a location 3,5 of said linear heptane chain,
R₂ is a second functional group at a location 3 of said first aryl group,
R₃ is a third functional group at a location 5 of said first aryl group,
R₄ is a fourth function group at a third location of said second aryl group, and OH is a hydroxyl functional group at a fifth location of said second aryl group

8. The method of claim 7 wherein
said white turmeric comprises alkaloids, terpenoids, flavonoids, steroids, saponins, tannins, phenols, phytosterols, gilycosides, protein amino acids, and volatile essential oils.

9. The method of claim 8 wherein said pre-cursor sub-fraction is defined by the formula: 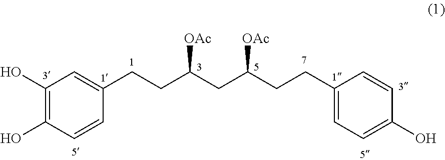 [SMILES: CC(=O)O[C@H](CCc1ccc(O)c(O)c1)C[C@H](CCc1ccc(O)cc1)OC(C)=O]
Wherein OAc is Acetoxyl functional group.

10. The method of claim 7 wherein said step (a) further comprises cleaning and drying said white turmeric rhizomes.

11. The method of claim 10 wherein said isolating and extracting of step (b) further comprising using a Soxhlet extractor with methanol as a solvent.

12. The method of claim 11 wherein said sub-fractions are suspended in water and partitioned with n-hexane to eliminate fatty impurities.

13. The method of claim 12 wherein said plurality of sub-fractions are obtained by a silica gel column chromatography using acetone-CHCl₃ gradient mixture.

14. The method of claim 13 wherein said step (c) further comprising selecting said sub-fractions having a inhibitory urease activities defined by half maximal inhibitory concentration (IC₅₀) of 9.6 μM against a urease enzyme and docking affinities with urease enzymes of −6.88 kcal/mol with said urease enzyme.

15. The method of claim 14 wherein said deacetylation reaction in step (d) further comprises performing a deacetylation reaction of said pre-cursor sub-fraction to obtain said plurality of sub-fractions.

16. The method of claim 15 wherein said optimal composition is defined by a formula:

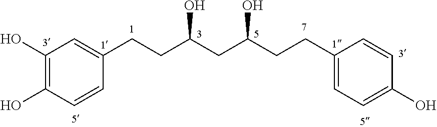 [SMILES: Oc1ccc(CC[C@H](O)C[C@H](O)CCc2ccc(O)c(O)c2)cc1]

17. The method of claim 7 wherein said deacetylation reaction of said precursor further comprises mixing Hydrochloric acid (HCl) with 36.5% strength and methanol (MeOH), wherein a weight or volume ratio (% w/w or % v/v) between MeOH and HCl is 30:1, wherein a weight or volume (% w/w or % v/v) between said precursor sub-fraction and methanol is 10:3.

18. The method of claim 17 further comprising stirring said mixture of HCl and MeOH at 200 rounds per minute at room temperature and for 24 hours.

19. The method of claim 18 wherein water is removed by using sodium sulfate (Na₂SO₄) to obtain a resultant sub-fraction.

20. The method of claim 19 further comprising performing column chromatography with CHCl₃:MeOH at volume or weight ratio (% w/w or % v/v) of 95:5 of said resultant sub-fraction to obtain said optimal sub-fraction.
